(19) United States
(12) Patent Application Publication
(10) Pub. No.: US 2009/0099074 A1
(43) Pub. Date: Apr. 16, 2009

(54) MODULATING FOOD INTAKE

(75) Inventors: Dominique P. Bridon, Franklin, CA (US); Karen Thibaudeau, Rosemere (CA); Alastair Ferguson, Kingston (CA); Denis Richard, Quebec (CA)

Correspondence Address: LOWRIE, LANDO & ANASTASI, LLP, ONE MAIN STREET, SUITE 1100, CAMBRIDGE, MA, 02142, US

(73) Assignee: CONJUCHEM BIOTECHNOLOGIES INC., Montreal (CA)

(21) Appl. No.: 11/965,288

(22) Filed: Dec. 27, 2007

Related U.S. Application Data
(60) Provisional application No. 60/879,871, filed on Jan. 10, 2007.

(51) Int. Cl. A61K 38/38; A61P 3/04

(52) U.S. Cl. 514/12

(57) ABSTRACT

The present disclosure relates to a conjugate comprising a PYY peptide or a functional derivative thereof which is coupled to a reactive group. Such a reactive group reacts with albumin so as to form a stable covalent bond therewith. The disclosure further provides methods of reducing water or food intake and reducing food intake between meals by administering such conjugates.

### CROSS-REFERENCE TO RELATED APPLICATIONS

The present application claims priority to U.S. Provisional Patent Application No. 60/879,871, filed on Jan. 10, 2007, the contents of which are incorporated herein by reference in their entirety.

### FIELD OF THE INVENTION

The present invention relates to compounds and methods for treating metabolic conditions or disorders. More particularly, the present relates to peptides, conjugates and methods for modulating food intake, modulating caloric availability and/or modulating water intake.

### BACKGROUND

A number of postprandial endocrine, paracrine or autocrine messenger products involved in the signaling of hunger and satiety that are present in circulation such as hormones or peptides. The results from an elevated or reduced plasma concentration of one or more of these products will have either global orexigenic or anorexigenic effects.

Examples of peptides associated with anorexigenic effects are pancreatic polypeptide (PP), neuropeptide Y (NPY) and peptide YY (PYY).

These peptides act through Y receptors for which five are known, Y1, Y2, Y3, Y4 and Y5 and regulate pancreatic secretion, gastric emptying and gastric motility. The Y receptors are found throughout the peripheral and central nervous systems as well as on various gastrointestinal organ cells.

Pancreatic polypeptide is secreted in the pancreas and helps control energy homeostasis through inhibition of pancreatic secretions such as for example insulin thus leading to an increased blood glucose level and signaling a need for reduced feeding.

Hypothalamic secreted neuropeptide Y participates in the control of food intake through binding and activation Y1 and possibly Y2 and Y5 receptors.

One of the most discussed examples in recent times is PYY₁₋₃₆. It is produced in endocrine L cells lining the distal small bowel and colon. The prepro PYY is clipped by signal peptidases to give proPYY₁₋₇₀. This peptide is further modified by prohormone dibasic convertase leading to PYY-Gly-Lys-Arg followed by Carboxypeptidase B to give PYY-Gly and finally to PYY₁₋₃₆ by amidation enzyme. It is then released from the cell where a metabolic derivative obtained through DPP-IV cleavage of the two N-terminal amino acids give circulating PYY₃₋₃₆.

PYY₁₋₃₆ binds and activates Y1, Y2 and Y5 receptors found on a variety of cells surfaces as for NPY. The cells are found peripherally in the gastrointestinal tract as well as on the arcuate nucleus. The result of interaction with the Y2 found on the arcuate is thought to lead to a central nervous system response. Alternatively, the Y2 receptors found peripherally on the surface of cell within the gastrointestinal tract have been shown to have an effect on gastric motility, gastric acid secretion and intestinal motility. The result of these interactions lead to reduced food and caloric intake.

Unlike PYY₁₋₃₆ which interacts equally with the Y1 and Y2 receptors, PYY₃₋₃₆ is selective to the Y2 receptor. A selective agonist of the Y2 receptor has been demonstrated to be beneficial as compared to a broad agonist. In fact, the Y1 receptor has been associated with hypertension (A. Balasubramaniam et al. J. Med. Chem. 2000, 43, 3420-27, Balasubramaniam A et al. Pept Res. 1988 1, 32-5). PYY₃₋₃₆ has been demonstrated to reduce food intake in vivo (Nature, 2002, 418, 650-4).

The advantage of using PYY₃₋₃₆ is that it is a natural appetite controlling hormone. There will not psychological side effect from the central nervous system such as when norepinephrine and serotonin reuptake inhibitor or other stimulants are used. Another advantage is that this class of therapeutic agent does not interfere with the absorption of certain nutritional or fat containing elements such as gastrointestinal lipase inhibitor that cause uncomfortable side effects. An inconvenience of using PYY₃₋₃₆ is need for multiple daily administrations.

### SUMMARY

PYY is an anorexigenic hormone that is cosecreted with glucagon-like peptide (GLP-1) by the endocrine cells of the distal digestive tract. PYY is released after meals and its effects seem to be both peripherally and centrally mediated. PYY acts of the central nervous system either via the vagus nerve or directly by crossing the blood brain barrier (BBB). Once conjugated to a blood component such as albumin, the PYY conjugate forms a large molecule unable to penetrate the BBB. It was found that PYY conjugates can act on the peripheral side of the BBB through, e.g., the area postrema (AP) and the subfomical organ (SFO) which are circumventricular organs known to express the Y2 receptors. These organs were found to play a role in the central action of PYY peptides. In addition, it was found that such conjugates reduced food intake, water intake and body weight gain for longer time periods than unconjugated PYY peptides.

According to one aspect, there is provided a compound comprising a PYY peptide or a functional derivative thereof which is coupled to a reactive group, the reactive group being capable of reacting with an amino group, a hydroxyl group or a thiol group on a blood component so as to form a stable covalent bond therewith, thereby substantially preventing the PYY peptide or functional derivative thereof from crossing the blood brain barrier.

According to another aspect, there is provided a conjugate comprising a blood component; and a PYY peptide or a functional derivative thereof which is coupled to a reactive group, wherein the reactive group is couples with at least an amino group, a hydroxyl group or a thiol group on the blood component so as to form a stable covalent bond therewith, thereby substantially preventing the PYY peptide or derivative thereof from crossing the blood brain barrier.

It should be understood that, in the compounds and conjugates of the present invention, the stable covalent bond between the PYY peptide or functional derivative thereof and the blood component can be formed in vivo or ex vivo.

According to another aspect, there is provided a method of enhancing, in a patient, the antiobesity activity of a PYY peptide or functional derivative thereof comprising covalently bonding the PYY peptide or functional derivative thereof to a blood component, thereby preventing the PYY peptide or functional derivative thereof from crossing the blood brain barrier when administered to the patient, wherein preventing the PYY peptide or functional derivative thereof from crossing the blood brain barrier results in an enhanced antiobesity activity of the PYY peptide or functional derivative thereof, e.g., as compared to the unconjugated PYY peptide or functional derivative thereof.

According to another aspect, there is provided in a method for treating a metabolic condition or disorder, e.g., such as a condition or disorder that can be alleviated by decreased caloric or nutrient availability, by administering a PYY peptide or a functional derivative thereof to a patient, the improvement wherein the PYY peptide or functional derivative thereof is covalently bonded to a blood component so as to prevent the PYY peptide or functional derivative thereof from crossing the blood brain barrier, thereby enhancing its activity. Such conditions and disorders include, but are not limited to, obesity, diabetes (e.g., type I diabetes, type 1.5 diabetes, type 2 diabetes, gestational diabetes), cardiovascular disorders, insulin resistance syndrome (Syndrome X), glucose intolerance, dyslipidemia, hypertension and tonicity disorders (e.g., hypotonicity disorders).

It should also be understood that, in the methods of the present invention, the covalent bonding between the PYY peptide or the functional derivative thereof and the blood component can be formed in vivo or ex vivo.

According to another aspect, there is provided a compound comprising a peptide of formula:

X₁-X₂-X₃-X₄-X₅-X₆-X₇-X₈-X₉-X₁₀-X₁₁-X₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆ (SEQ ID NO: 1)

wherein, X₁ is absent, tyr or ala; X₂ is absent or pro; X₃ is absent, lys or an analog thereof, ile, leu, or ala; X₄ is absent, lys or an analog thereof, or glu; Xs is absent or pro; X₆ is absent, glu, val or asp; X₇ is absent, ala, tyr or asn; X₈ is absent or pro; X₉ is absent or gly; X₁₀ is absent, glu or asp; X₁₁ is absent, asp or asn; X₁₂ is absent, lys or an analog thereof, or ala; X₁₃ is absent, lys or an analog thereof, ser, thr, or pro; X₁₄ is absent, lys or an analog thereof, ala, or pro; X₁₅ is absent, lys or an analog thereof, or glu; X₁₆ is absent, glu, gin or asp; X₁₇ is absent, leu or met; X₁₈ is absent, lys or an analog thereof, ser, or ala; X₁₉ is absent, arg or gln; X₂₀ is absent or tyr; X₂₁ is absent, tyr or ala; X₂₂ is ala or ser; X₂₃ is ser, asp or ala; X₂₄ is leu; X₂₅ is arg or lys; X₂₆ is his, arg or lys; X₂₇ is tyr; X₂₈ is leu or ile; X₂₉ is asn; X₃₀ is leu or met; X₃₁ is val, leu or ile; X₃₂ is thr; X₃₃ is arg or lys; X₃₄ is gin or pro; X₃₅ is arg or lys; X₃₆ is tyr or a derivative thereof; and A is absent lys or a derivative thereof, and at least one reactive group coupled to any one of X₁ to X₃₆ and A, directly or via a linking group. Such compounds can be used, e.g., in a method described herein.

According to another aspect, there is provided a conjugate comprising a blood component and a compound having a peptide of formula:

X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆ (SEQ ID NO: 1)

wherein, X₁ is absent, tyr or ala; X₂ is absent or pro; X₃ is absent, lys or an analog thereof, ile, leu, or ala; X₄ is absent, lys or an analog thereof, or glu; X₅ is absent or pro; X₆ is absent, glu, val or asp; X₇ is absent, ala, tyr or asn; X₈ is absent or pro; X₉ is absent or gly; X₁₀ is absent, glu or asp; X₁₁ is absent, asp or asn; X₁₂ is absent, lys or an analog thereof, or ala; X₁₃ is absent, lys or an analog thereof, ser, thr, or pro; X₁₄ is absent, lys or an analog thereof, ala, or pro; X₁₅ is absent, lys or an analog thereof, or glu; X₁₆ is absent, glu, gin or asp; X₁₇ is absent, leu or met; X₁₈ is absent, lys or an analog thereof, ser, or ala; X₁₉ is absent, arg or gin; X₂₀ is absent or tyr; X₂₁ is absent, tyr or ala; X₂₂ is ala or ser; X₂₃ is ser, asp or ala; X₂₄ is leu; X₂₅ is arg or lys; X₂₆ is his, arg or lys; X₂₇ is tyr; X₂₈ is leu or ile; X₂₉ is asn; X₃₀ is leu or met; X₃₁ is val, leu or ile; X₃₂ is thr; X₃₃ is arg or lys; X₃₄ is gin or pro; X₃₅ is arg or lys; X₃₆ is tyr or a derivative thereof, and a reactive group coupled to any one of X.sub.1-X.sub.36 and A, directly or via a linking group, and wherein the reactive group couples with at least an amino group, a hydroxyl group or a thiol group on the blood component so as to form a stable covalent bond therewith. Such conjugates can be used, e.g., in the methods described herein.

It has been found that the compounds and conjugates described herein demonstrate an enhanced anti-obesity activity with respect to PYY peptides such as PYY₁₋₃₆, PYY₃₋₃₆ and PYY₂₂₋₃₆. It also has been found that these compounds and conjugates are efficient for reducing the food consumption, body weigh gain and/or water intake of a subject, thereby treating or preventing metabolic conditions or disorders, e.g., which can be alleviated by decreased caloric availability. Such methods can be used, e.g., to reduce food intake, body weight gain and/or water intake after food deprivation (e.g., after being on a diet of reduced food intake). The food deprivation can be, e.g., for at least about 1 day, 2 days, 3 days, 4 days, 5 days, 6 days, one week, one and a half weeks, two weeks, two and a half weeks, three weeks, three and a half weeks, and one month. In some embodiments, the compound or conjugate, e.g., a compound or conjugate described herein, can be administered prior to food intake in a subject that has been food deprived. Such methods can also be used, e.g., to reduce food intake (e.g., meal proportion size) during refeeding (i.e., after a standard normal between meal period, e.g., 3 to 8 hours). In some embodiments, the compound or conjugate, e.g., the compound or conjugate described herein, can be administered before food intake.

It has been found that the methods of the present invention are effective for enhancing the antiobesity activity of PYY peptide or a derivative thereof and/or for treating obesity. It also has been found that by preventing the compounds or conjugates from crossing the blood brain barrier, an enhanced antiobesity activity of the PYY peptides or derivative thereof was observed.

The expression “a PYY peptide or a functional derivative thereof” as used herein refers to a PYY peptide such as PYY₁₋₃₆ or PYY₃₋₃₆ or to a functional derivative of the PYY peptide such as PYY₂₂₋₃₆. Such a functional derivative would be understood by a person skilled in the art as a derivative which substantially maintains the activity of the PYY peptide. Preferably, such a functional derivative has an in vitro NPY Y2 receptor binding activity which is at least 1/100 of the in vitro NPY Y2 receptor binding activity of PYY₃₋₃₆. More preferably, the functional derivative has an in vitro NPY Y2 receptor binding activity which is equal or superior to the in vitro NPY Y2 receptor binding activity of PYY₃₋₃₆. In a non-limitative manner, the functional derivative can comprise a peptide of the following formula: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-A (SEQ ID NO: 1) or Z₁-Z₂-Z₃-Z₄-Z₅-Z₆-Z₇-Z₈-Z₉-Z₁₀-Z₁₁-Z₁₂ (SEQ ID NO: 2) wherein X₁ to X₃₆, and A are as previously defined, and wherein Z₁ is ala, Z₄ is arg, Z₈ is asn, Z₁₂ is arg, and Z₂, Z₃, Z₅ to Z₇ and Z₉ to Z₁₁ are selected from the group consisting of the natural amino acids.

The expression “lys or an analog thereof” refers to a lysine or an analog thereof that will substantially maintains the activity of the peptide. In a non-limitative manner, the lys analog can be of formula:

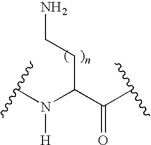

where n is an integer having a value of 0, 1, 2, 3 or 4.

The expression “tyr or a derivative thereof” refers to a tyrosine or a derivative thereof that will substantially maintains the activity of the peptide. In a nonlimitative manner, the tyr derivative can be of formula:

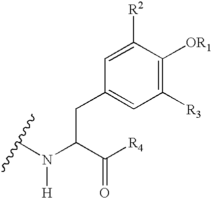

where

R₁ is H, a protecting group (PG), a C₁-C₁₀ branched, linear or cyclic alkyl, a phosphate or a sulfate; R₂ and R₃ are same or different and selected from the group consisting of H, D and I; and R₄ is OH, OPG, OR₅, SH, SPG, SR₅, NH₂, NHPG, N(PG)₂, N(R₅)₂, NR₅PG, or NHR₆, where R₅ is a C₁-C₁₀ branched, linear or cyclic alkyl, and R₆ is a solid phase support.

The expression “protecting group (PG)” as used herein refers to suitable protecting groups as defined in T. W. Greene and P. G. M. Wuts, Protective Groups in Organic Synthesis, 3rd Edition, (1999) John Wiley & Sons, which is hereby incorporated by reference. The person skilled in the art will understand that nature of the protecting group will vary according to the functionality that has to be protected. Greene et al. discloses, as example, various protecting groups for carboxylic acids, alcohols, thiols, amines, amides etc.

The expression “lys or a derivative thereof” refers to a lysine or a derivative thereof that will substantially maintains the activity of the peptide. In a non-limitative manner, the lys derivative can be of formula:

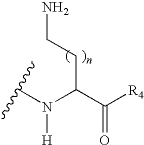

where

R₄ is OH, OPG, OR₅, SH, SPG, SR₅, NH₂, NHPG, N(PG)₂, N(R₅)₂, NR₅PG, or NHR₆, where R₅ is a C₁-C₁₀ branched, linear or cyclic alkyl, and R₆ is a solid phase support; and

n is an integer having a value of 0, 1, 2, 3 or 4.

The PYY peptide or functional derivative thereof can be selected from SEQ IDS NO: 1 to 15, preferably from SEQ IDS NO: 3 to 13, and more preferably from SEQ ID NO: 6, 12 or 13.

In the compounds and conjugates, there is preferably only one reactive group. Advantageously, the reactive group is coupled to any one of X₁ to X₂₁, X₂₃, X₂₄, X₂₆ to X₂₈, X₃₀ to X₃₂, X₃₄ to X₃₆, and A of SEQ ID NO:1. Alternatively, the reactive group can be coupled to any one of Z₁ to Z₁₂ and preferably to any one of Z₂, Z₃, Z₅ to Z₇ and Z₉ to Z₁₁ of SEQ ID NO:2. The reactive group can also be connected to the peptide or the PYY functional derivative by means of a linking group.

According to preferred embodiments, in the compounds and conjugates which comprise a peptide of formula: X₁-X₂-X₃-X₄-X₅-X₆-X₇-X₈-X₉-X₁₀-X₁₁-X₁₂-X₁₃-X₁₄-X₁₅-X₁₆-X₁₇-X₁₈-X₁₉-X₂₀-X₂₁-X₂₂-X₂₃-X₂₄-X₂₅-X₂₆-X₂₇-X₂₈-X₂₉-X₃₀-X₃₁-X₃₂-X₃₃-X₃₄-X₃₅-X₃₆-A (SEQ ID NO: 1):

X₁ can be absent, the reactive group, linking group-(reactive group), tyr or ala, the tyr or ala being optionally coupled to the reactive group or to the linking group-(reactive group). Preferably, X₁ is absent. X₂ can be absent, pro, the reactive group or the linking group-(reactive group). Preferably, X₂ is absent. X₃ can be absent, lys or an analog thereof, (reactive group)-lys, (reactive group)-linking group-lys, (reactive group)-lys analog, (reactive group)-linking group-lys analog, ile, leu, or ala, wherein the reactive group is coupled to the free amine of lys or lys analog. Preferably, X₃ is leu. X₄ can be absent, lys or an analog thereof, (reactive group)-lys, (reactive group)-linking group-lys, (reactive group)-lys analog, (reactive group)-linking group-lys analog, or glu, wherein the reactive group is coupled to the free amine of lys or lys analog. Preferably, X₄ is lys (reactive group)-lys or (reactive group)-linking group-lys. X₅ is preferably pro. X₆ is preferably glu. X₇ is preferably ala. X₈ is preferably pro. X₉ is preferably gly. X₁₀ is preferably glu. X₁₁ is preferably asp. X₁₂ can be absent, lys or an analog thereof, (reactive group)-lys, (reactive group)-linking group-lys, (reactive group)-lys analog, (reactive group)-linking group-lys analog, or ala, wherein the reactive group is coupled to the free amine of lys or lys analog. Preferably, X₁₂ is ala. X₁₃ can be absent, lys or an analog thereof, (reactive group)-lys, (reactive group)-linking group-lys, (reactive group)-lys analog, (reactive group)-linking group-lys analog, ser, thr, or pro, wherein the reactive group is coupled to the free amine of lys or lys analog. Preferably, X₁₃ is ser. X₁₄ can be absent, lys or an analog thereof, (reactive group)-lys, (reactive group)-linking group-lys, (reactive group)-lys analog, (reactive group)-linking group-lys analog, ala or pro, wherein the reactive group is coupled to the free amine of lys or lys analog. X₁₄ is preferably pro. X₁₅ can be absent, lys or an analog thereof, (reactive group)-lys, (reactive group)-linking group-lys, (reactive group)-lys analog, (reactive group)-linking group-lys analog, or glu, wherein the reactive group is coupled to the free amine of lys or lys analog. X₁₅ is preferably glu. X₁₆ is preferably glu. X₁₇ is preferably leu. X₁₈ can be absent, lys or an analog thereof, (reactive group)-lys, (reactive group)-linking group-lys, (reactive group)-lys analog, (reactive group)-linking group-lys analog, ser, or ala, wherein the reactive group is coupled to the free amine of lys or lys analog. X₁₈ is preferably ser. X₁₉ is preferably arg. X₂₀ is preferably tyr. X₂₁ can be absent, tyr, ala, a reactive group, or linking group-(reactive group), wherein the linking group is coupled to X₂₀ and X₂₂. X₂₁ is preferably tyr. X₂₂ is preferably ala. X₂₃ is preferably ser. X₂₅ is preferably arg. X₂₆ is preferably his. X₂₈ is preferably leu. X₃₀ is preferably leu. X₃₁ is preferably val. X₃₃ is preferably arg. X₃₄ is preferably gln. X₃₅ is preferably arg. X₃₆ is preferably tyr.

In a preferred embodiment, the reactive group can be selected from the group consisting of Michael acceptors (preferably an unsaturated carbonyl such as a vinyl carbonyl or a vinyl sulfone moiety), succinimidyl-containing groups (such as, N-hydroxysuccinimide (NHS), N-hydroxy-sulfosuccinimide (sulfo-NHS) etc.), an electrophilic thiol acceptor (such as pyridyldithio (Pyr-S-S), an alpha halogenated alkyl carbonyl (such as an alpha halogenated alkyl carbonyl where the alkyl, further to the halogen substituent, may contains or not a substituent such as a C₁-C₈ alkyl or phenyl), and maleimido-containing groups (such as gamma-maleimide-butyralamide (GMBA), beta-maleimidopropionic acid (MPA), alpha-maleimidoacetic acid (MAA) etc.). Advantageously, the reactive group is a maleimido-containing group. Alternatively, the reactive group is advantageously an alpha halogenated alkyl carbonyl and preferably alpha iodo acetyl. Preferably, the reactive group is a reactive group, which is capable of reacting with an amino group, a hydroxyl group or a thiol group on a blood component so as to form a stable covalent bond.

As example, the maleimido group is most selective for sulfhydryl groups on peptides when the pH of the reaction mixture is kept between 6.5 and 7.4. At pH 7.0, the rate of reaction of maleimido groups with sulfhydryls is 1000-fold faster than with amines. A stable thioether linkage between the maleimido group and the sulfhydryl is formed which cannot be cleaved under physiological conditions. Primary amines can be the principal targets for NHS esters. Accessible α-amine groups present on the N-termini of proteins can react with NHS esters. However, α-amino groups on a protein may not be desirable or available for the NHS coupling. While five amino acids have nitrogen in their side chains, only the ε-amine of lysine reacts significantly with NHS esters. An amide bond can be formed when the NHS ester conjugation reaction reacts with primary amines releasing N hydroxysuccinimide.

In a preferred embodiment, the reactive group is coupled to an amino acid of the peptide via a linking group (or linker). A linking group that reacts with the reactive group and the amino acid of the peptide includes, but not limited to (2-amino)ethoxy acetic acid (AEA), ethylenediamine (EDA), amino ethoxy ethoxy succinimic acid (AEES), AEES-AEES, 2-[2-(2-amino)ethoxy)]ethoxy acetic acid (AEEA), AEEA-AEEA, —NH₂—(CH₂)ₙ—COOH where n is an integer between 1 and 20 and alkyl chain (C₁-C₁₀) motif saturated or unsaturated in which could be incorporated oxygen nitrogen or sulfur atoms, such as, but not limited to glycine, 3-aminopropionic acid (APA), 8-aminooctanoic acid (OA) and 4-aminobenzoic acid (APhA) and combinations thereof.

In a preferred embodiment, the blood component is a blood protein, more preferably is albumin (such as human albumin).

Preferably, the invention relates to compounds and conjugates that include PYY₃₋₃₆ or derivatives thereof, which can be shortened versions of the latter such as PYY₂₂₋₃₆. The conjugates formed by the ex vivo, in vivo or in vitro covalent bonding between the peptides of the present invention and a blood component have been found to be very selective to the neuropeptide Y2 receptor.

PP and NPY peptides could also be suitable as an alternative to PYY or its functional derivatives in the various embodiments of the present invention.

The methods described herein include extending the effective therapeutic life of the conjugated peptide derivatives as compared to administration of the unconjugated peptide to a patient. Moreover, the activity of the conjugated peptide derivatives described herein is considerably enhanced as compared the unconjugated peptide to a patient. The derivatives or modified peptides can be of a type designated as a DAC™ (Drug Affinity Complex), which comprises the peptide molecule and a linking group together with a chemically reactive group capable of reaction with a reactive functionality of a mobile blood protein. By reaction with the blood component or protein the modified peptide, or DAC, may be delivered via the blood to appropriate sites or receptors. Moreover, conjugating the peptides to a blood component can provide protection against the degradation of enzymes.

A. Specific Labeling.

Preferably, the compounds, derivatives or modified peptides are designed to specifically react with thiol groups on mobile blood proteins. Such a reaction is preferably established by covalent bonding of the peptide modified with a maleimido-containing group linked to a thiol group on a mobile blood protein such as albumin or IgG.

Under certain circumstances, specific labeling with maleimido-containing group offers several advantages over non-specific labeling of mobile proteins with groups such as NHS and sulfo-NHS. Thiol groups are less abundant in vivo than amino groups. Therefore, the compounds described herein such as maleimido-modified peptides, can covalently bond to fewer proteins. For example, in albumin (an abundant blood protein) there is only a single thiol group. Thus, peptide-(maleimido-containing group)-albumin conjugates can tend to comprise a 1:1 molar ratio of peptide to albumin. In addition to albumin, IgG molecules (class II) also have free thiols. Since IgG molecules and serum albumin make up the majority of the soluble protein in blood they also make up the majority of the free thiol groups in blood that are available to covalently bond to maleimide-modified peptides.

Further, even among free thiol-containing blood proteins, including IgGs, specific labeling with a maleimido-containing group leads to the preferential formation of peptide-(maleimido-containing group)-albumin conjugates, due to the unique characteristics of albumin itself. The single free thiol group of albumin, highly conserved among species, is located at amino acid residue 34 (Cys34). It has been demonstrated recently that the Cys34 of albumin has increased reactivity relative to free thiols on other free thiol-containing proteins. This is due in part to the very low pK value of 5.5 for the Cys34 of albumin. This is much lower than typical pK values for cysteine residues in general, which are typically about 8. Due to this low pK, under normal physiological conditions Cys34 of albumin is predominantly in the anionic form, which dramatically increases its reactivity. In addition to the low pK value of Cys34, another factor, which enhances the reactivity of Cys34 is its location in a crevice close to the surface of one loop of region V of albumin. This location makes Cys34 very available to ligands of all kinds, and is an important factor in Cys34's biological role as a free radical trap and a free thiol scavenger. These properties make Cys34 highly reactive toward maleimidepeptides, and the reaction rate acceleration can be as much as 1000-fold relative to rates of reaction of maleimide-peptides with other free-thiol containing proteins.

Another advantage of peptide(maleimido-containing group)-albumin conjugates is the reproducibility associated with the 1:1 loading of peptide to albumin specifically at Cys34. Other techniques, such as glutaraldehyde, DCC, EDC and other chemical activations of, e.g., free amines, lack this selectivity. For example, albumin contains 52 lysine residues, 25 to 30 of which are located on the surface of albumin and therefore accessible for conjugation. Activating these lysine residues, or alternatively modifying peptides to couple through these lysine residues, results in a heterogeneous population of conjugates. Even if statistical 1:1 molar ratios of peptide to albumin are employed, the yield will consist of multiple conjugation products, some containing 0, 1, 2 or more peptides per albumin, and each having peptides randomly coupled at any one or more of the 25 to 30 available lysine sites. Given the numerous possible combinations, characterization of the exact composition and nature of each conjugate batch becomes difficult, and batch-to-batch reproducibility is all but impossible, making such conjugates less desirable as a therapeutic. Additionally, while it would seem that conjugation through lysine residues of albumin would at least have the advantage of delivering more therapeutic agent per albumin molecule, studies have shown that a 1:1 ratio of therapeutic agent to albumin is preferred. In an article by Stehle, et al., “The Loading Rate Determines Tumor Targeting properties of Methotrexate-Albumin Conjugates in Rats,” Anti-Cancer Drugs, Vol. 8, pp. 677-685 (1988), the authors report that a 1:1 ratio of the anti-cancer methotrexate to albumin conjugated via amide coupling of one of the available carboxylic acids on methotrexate to any lysine on albumin gave the most promising results. The conjugates describe therein were preferentially taken up by tumor cells, whereas the conjugates bearing 5:1 to 20:1 methotrexate molecules to albumin had altered HPLC profiles and were quickly taken up by the liver in vivo. It is postulated that at these higher ratios, confer conformational changes to albumin diminishing its effectiveness as a therapeutic carrier.

Through controlled administration of maleimido-peptides in vivo, one can control the specific labeling of albumin and IgG in vivo. In typical administrations, 80-90% of the administered maleimido-peptides will label albumin and less than 5% will label IgG. Trace labeling of free thiols such as glutathione, cysteine or Cys-Gly will also occur. Such specific labeling is preferred for in vivo use as it permits an accurate calculation of the estimated half-life of the administered agent.

In addition to providing controlled specific in vivo labeling, maleimide peptides can provide specific labeling of serum albumin and IgG ex vivo. Such ex vivo labeling involves the addition of maleimide-peptides to blood, serum or saline solution containing serum albumin and/or IgG. Once conjugation has occurred ex vivo with the maleimido-peptides, the blood, serum or saline solution can be readministered to the patient's blood for in vivo treatment.

In contrast to NHS-peptides, maleimido-peptides are generally quite stable in the presence of aqueous solutions and in the presence of free amines. Since maleimido-peptides will only react with free thiols, protective groups are generally not necessary to prevent the maleimido-peptides from reacting with itself. In addition, the increased stability of the modified peptide permits the use of further purification steps such as HPLC to prepare highly purified products suitable for in vivo use. Lastly, the increased chemical stability provides a product with a longer shelf life.

B. Non-Specific Labeling.

The peptides described herein may also be modified for non-specific labeling of blood components. Bonds to amino groups will also be employed, particularly with the formation of amide bonds for non-specific labeling. To form such bonds, one may use as a chemically reactive group a wide variety of active carboxyl groups, particularly esters, where the hydroxyl moiety is physiologically acceptable at the levels required. While a number of different hydroxyl groups may be employed in these linking agents, the most convenient would be N-hydroxysuccinimide (NHS) and N-hydroxy-sulfosuccinimide (sulfo-NHS).

Other linking agents that may be utilized are described in U.S. Pat. No. 5,612,034, which is hereby incorporated by reference. The various sites with which the chemically reactive group of the modified peptides may react in vivo include cells, particularly red blood cells (erythrocytes) and platelets, and proteins, such as immunoglobulins, including IgG and IgM, serum albumin, ferritin, steroid binding proteins, transferrin, thyroxin binding protein, α-2-macroglobulin, and the like. Those receptors with which the modified peptides react, which are not long-lived, will generally be eliminated from the human host within about three days. The proteins indicated above (including the proteins of the cells) will remain at least three days, and may remain five days or more (usually not exceeding 60 days, more usually not exceeding 30 days) particularly as to the half life, based on the concentration in the blood.

For the most part, reaction can be with mobile components in the blood, particularly blood proteins and cells, more particularly blood proteins and erythrocytes. By “mobile” is intended that the component does not have a fixed situs for any extended period of time, generally not exceeding 5 minutes, more usually one minute, although some of the blood component may be relatively stationary for extended periods of time.

Initially, there will be a relatively heterogeneous population of functionalized proteins and cells. However, for the most part, the population within a few days will vary substantially from the initial population, depending upon the half-life of the functionalized proteins in the blood stream. Therefore, usually within about three days or more, IgG will become the predominant functionalized protein in the blood stream.

Usually, by day 5 post-administration, IgG, serum albumin and erythrocytes will be at least about 60 mole %, usually at least about 75 mole %, of the conjugated components in blood, with IgG, IgM (to a substantially lesser extent) and serum albumin being at least about 50 mole %, usually at least about 75 mole %, more usually at least about 80 mole %, of the non-cellular conjugated components.

The desired conjugates of non-specific modified peptides to blood components may be prepared in vivo by administration of the modified peptides to the patient, which may be a human or other mammal. The administration may be done in the form of a bolus or introduced slowly over time by infusion using metered flow or the like.

If desired, the subject conjugates may also be prepared ex vivo by combining blood with modified peptides of the present invention, allowing covalent bonding of the modified peptides to reactive functionalities on blood components and then returning or administering the conjugated blood to the host. Moreover, the above may also be accomplished by first purifying an individual blood component or limited number of components, such as red blood cells, immunoglobulins, serum albumin, or the like, and combining the component or components ex vivo with the chemically reactive modified peptides. The functionalized blood or blood component may then be returned to the host to provide in vivo the subject therapeutically effective conjugates. The blood also may be treated to prevent coagulation during handling ex vivo.

Other sources of blood components, such as recombinant proteins are also suitable for the preparation of the conjugates described herein. For example, recombinant albumin can be used, e.g., in vitro, to prepare a conjugate described herein. In some embodiments, the albumin is MEDWAY® (ALBREC®, GB-1057, Mitsubishi Tanabe Pharma Corp., Osaka, Japan). MEDWAY is a recombinant human albumin (rHA) that is produced in vitro using recombinant yeast technology, in which genetically modified yeast (Pichia pastoris) secrete soluble rHA which can be subsequently harvested, purified and formulated for the indicated treatment.

Some of the preferred compounds of the invention are derivatives of PYY₁₋₃₆ and PYY₃₋₃₆. These derivatives comprise a strategically placed maleimido-containing group as described above. PYY₁₋₃₆ and PYY₃₋₃₆ have the following structures:

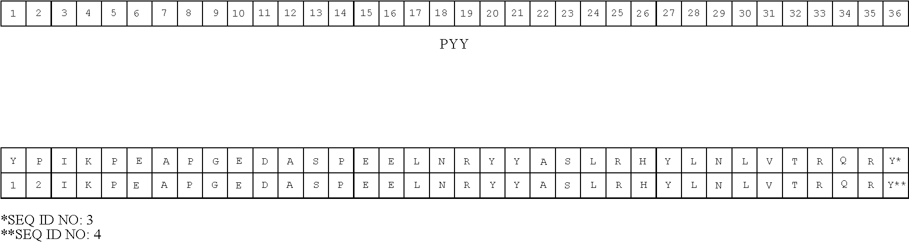

These peptides have an alpha helical structure starting at position 18 running through to position 36 (example on PYY in Biochemistry, 2000, 39, 9935). The amino acids at positions 22, 25, 29 and 33 can be considered as relatively important for the activity. All derivatives of these peptides can be truncated, modified, mutated or intact peptides. Preferably, they are able to expose side chains found on these four amino acid residues. These residues are conserved in PYY₁₋₃₆ (SEQ ID NO: 3), PYY₃₋₃₆ (SEQ ID NO: 4), pancreatic polypeptide and neuropeptide Y. Secondary conserved amino acids of potential importance can be those at positions 5, 8, 9, 12, 15, 20, 24, 27, 32, 35 and 36.

The compounds and conjugates described herein can be used to treat metabolic conditions and disorders such as conditions and disorders that can be alleviated by reduced caloric and/or nutrient availability. As used herein, the term “metabolic conditions and disorders that can be alleviated by reduced caloric and/or nutrient availability” refer to conditions and disorders caused by or complicated by or aggravated by a relatively high nutrient availability or that can be alleviated by reduced caloric or nutrient availability. Such conditions or disorders include, but are not limited to, obesity, diabetes (e.g., type 1 diabetes, type 1.5 diabetes, type 2 diabetes, gestational diabetes), eating disorders, insulin resistance syndromes (e.g., Syndrome X), glucose intolerance, dyslipidemia (e.g., LDL cholesterol, triglyceride levels and/or HDH levels), metabolic syndrome and tonicity disorders (e.g., hypotonicity, hyponatremia).

A metabolic syndrome as used herein refers to subjects having three or more of the following criteria: (1) waist circumference of greater than 102 cm for men and greater than 88 cm for women; (2) serum triglycerides of greater than 1.7 mmol/l; (3) blood pressure of greater than 130/85 mmHg; (4) HDL-cholesterol of less than 1.0 mmol/l in men and less than 1.3 mmol/l in women; and (5) serum glucose of greater than 6.1 mmol/l (greater than 5.6 mmol/1 may be applicable). In addition, a subject having diabetes or impaired fasting glucose (IFG) or impaired glucose tolerance (IGT) or insulin resistance (assessed by clamp studies), plus at least two of the following criteria: (1) waist-to-hip ratio of greater than 0.90 in men or greater than 0.85 in women; (2) serum triglycerides of greater than 1.7 mmol/l or HDL-cholesterol of less than 0.9 mmol/l in men and less than 1.0 mmol/l in women; (3) blood pressure of greater than 140/90 mmHg; (4) urinary albumin excretion rate of greater than 20 micrograms/minute or albumin:creatinine ratio of greater than 30 mg/g, has metabolic syndrome.

In one aspect, the invention provides a method of treating obesity in an obese or overweight subject by administering a therapeutically effective amount of a compound or conjugate described herein. While “obesity” is generally defined as a body mass index over 30, for purposes of this disclosure, any subject, including those with a body mass index of less than 30, who needs or wishes to reduce body weight is included in the scope of “obese.” A class of subjects having a body mass index of less than 30 that can be treated by the methods described herein are subjects that are overweight. Subjects who are insulin resistant, glucose intolerant, or have any form of diabetes mellitus (e.g., type 1, 2 or gestational diabetes) can benefit from this method. Obesity in the context of a body mass index over 30 is a recognized risk factor for cardiovascular disorders including hypertension, atherosclerosis, congestive heart failure; stroke; gallbladder disease; osteoarthritis; sleep apnea; reproductive disorders such as polycystic ovarian syndrome.

In some embodiments, the subject is obese but neither diabetic nor prediabetic; obese and diabetic or pre-diabetic; obese but not affected by metabolic syndrome; obese and affected by the metabolic syndrome; overweight but neither diabetic nor prediabetic; overweight and diabetic or prediabetic; overweight but not affected by metabolic syndrome; overweight and affected by metabolic syndrome; affected by metabolic syndrome but neither diabetic nor prediabetic (depending on the definition of metabolic syndrome); affected by metabolic syndrome but neither obese nor overweight.

In another aspect, the invention provides a method of treating a disorder or condition associated with water imbalance such as a condition or disorder associated with excess water intake or hypnotically. The method includes administering a therapeutically effective amount of a compound or conjugate described herein. Excess water intake refers to water intake above normal water excretory capacity (e.g., about 12 L/day to 20 L/day).

Another aspect of the invention is to reduce excess intestinal water and decreasing excess electrolyte secretion.

Another aspect of the invention is to relieve tumor necrosis factor (TNF)-induced acute pancreatitis through the inhibition of NF-B translocation to acinar nuclei (Vona-Davis L. et al., J. Am. Coll. Surg., 2004, 199, 87-95) using the DAC PYY₁₋₃₆ series of derivatives.

The compounds and conjugates can be administered according to any technique deemed suitable by one of skill in the art. For example, the compounds and conjugates can be administered by any of the following means: (a) enterally, e.g., orally (by mouth), rectally (e.g., in the form of a suppository or an enema), by feeding tube (e.g., gastric feeding tube, duodenal feeding tube, gastrostromy); (b) parenterally, e.g., subcutaneously, intravenously, intramuscularly, intradermally (into the skin itself), transdermally (diffusion through skin, e.g., intact skin), intra-arterially, intra-peritoneally, intracardiac (into the heart) administration, intraosseous (into the bone marrow) administration intrathecally (into the spinal canal), transmucosally (diffusion through a mucous membrane, e.g., insufflation (snorting), nasally, e.g., intranasally), sublingually (under the tongue), buccally (through the cheek), vaginally, by inhalation (e.g., pulmonary administration); and (c) topically; (d) epidurally (injection or infusion into the epidural space; (e) intravitreally. Each administration of the compounds and conjugates can be by bolus or by infusion. In preferred embodiments, the compound or conjugate is administered subcutaneously. In a particular embodiment, the compound or conjugate is administered subcutaneously using a needle, e.g., a 28-gauge needle, a 29-gauge needle, a 30-gauge needle, a 31-gauge needle, a 32-gauge needle, or a 33-gauge needle, or a larger gauge needle.

### BRIEF DESCRIPTION OF DRAWINGS

Further features and advantages of the invention will become more readily apparent from the following description of preferred embodiments as illustrated by way of examples in the appended drawings wherein:

FIG. 1 is a diagram showing a comparison between the anti-obesity activity of PYY₃₋₃₆ and the anti-obesity activity a compound according to a preferred embodiment of the invention, wherein the anti-obesity activity of these compounds has been determined in an experiment by administering them, at various doses, to Sprague-Dawley rats and by measuring the food consumption of these rats before and after administration of these compounds;

FIG. 2 is another diagram as in FIG. 1, wherein the PYY peptide and the compound according to a preferred embodiment of the invention have been administered to the rats according to other dosages;

FIG. 3 is a diagram showing the reduction in food intake after 24 hours, which has been generated by the administration of the PYY peptide and the compound of the invention, during the experiment described in FIG. 1;

FIG. 4 is a diagram showing the reduction in food intake after 24 hours, which has been generated by the administration of the PYY peptide and the compound of the invention, during the experiment described in FIG. 2; and

FIG. 5 is a plot showing the influence of the dosage of another compound according to a preferred embodiment of the invention on the total food intake of Sprague-Dawley rats over time.

FIG. 6 is a diagram showing body weight gain and water intake during refeeding after subcutaneous administration of (A) control (saline) and PYY or (B) control (saline) and albumin conjugated PYY for sham operated rats.

FIG. 7 is a diagram showing food intake and water intake during refeeding after subcutaneous administration of (A) control (saline) and PYY or (B) control (saline) and albumin conjugated PYY for sham operated and AP-SFO lesioned rats.

FIG. 8 is a diagram showing C-fos mRNA 1 hour after subcutaneous injections of PYY or albumin conjugated PYY for sham operated and AP-SFO lesioned rats.

Other features and advantages of the invention will be apparent from the following detailed description, and from the claims.

### DESCRIPTION OF PREFERRED EMBODIMENTS

This invention is further illustrated by the following examples that should not be construed as limiting. The contents of all references, patents and published patent applications cited throughout this application are incorporated herein by reference.

### EXAMPLES

### 1. Synthetic Scheme

General

The synthesis of the PYY peptides and functional derivatives thereof was performed using an automated solid-phase procedure on a Symphony Peptide Synthesizer with manual intervention during the generation of the DAC peptide. The synthesis was performed on Fmoc-protected Ramage amide linker resin, using Fmoc-protected amino acids. Coupling was achieved by using O-benzotriazol-1-yl-N,N,N′,N′-tetramethyl-uronium hexafluorophosphate (HBTU) and diisopropylethylamine (DIEA) as the activator cocktail in N,N-dimethylformamide (DMF) solution. The Fmoc protective group was removed using 20% piperidine/DMF. When needed, a Boc-protected amino acid was used at the N-terminus in order to generate the free N_{α}-terminus after the peptide was cleaved from resin. All amino acids used during the synthesis possessed the L-stereochemistry unless otherwise stated. Sigmacoted glass reaction vessels were used during the synthesis.

Compound I (PYY₃₋₃₆)

(SEQ ID NO: 4)

Ile-Lys-Pro-Glu-Ala-Pro-Gly-Glu-Asp-Ala-Ser-Pro-
Glu-Glu-Leu-Asn-Arg-Tyr-Tyr-Ala-Ser-Leu-Arg-His-
Tyr-Leu-Asn-Leu-Val-Thr-Arg-Gln-Arg-Tyr-CONH₂

Step 1: Solid phase peptide synthesis of the DAC™ peptide on a 100 μmole scale was performed using manual and automated solid-phase synthesis, a Symphony Peptide Synthesizer and Ramage resin. The following protected amino acids were sequentially added to resin. Fmoc-Tyr(tBu)-OH, Fmoc-Arg(Pbf)-OH, Fmoc-Cln(Trt)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Trt)-OH, Fmoc-Arg(Pbf)-OH, Fmoc-Leu-OH, Fmoc-Ser(tBu)-OH, Fmoc-Ala-OH, Fmoc-Tyr-(tBu)-OH, Fmoc-Tyr(tBu)-OH, Fmoc-Arg(Pbf)-OH, Fmoc-Asn(Trt)-OH, Fmoc-Leu-OH, Fmoc-Glu(tBu)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The peptide was cleaved from the resin using 85% TFA/5% TIS/5% thioanisole and 5% phenol, followed by precipitation by dry-ice cold (0-4° C.) Et₂O. The crude peptide was collected on a polypropylene sintered funnel, dried, redissolved in a 40% mixture of acetonitrile in water (0.1% TFA) and lyophilized to generate the corresponding crude material used in the purification process.

Compound II

(SEQ ID NO: 5)

Ile-Lys-Pro-Glu-Ala-Pro-Gly-Glu-Asp-Ala-Ser-Pro-
Glu-Glu-Leu-Asn-Arg-Tyr-Tyr-Ala-Ser-Leu-Arg-His-
Tyr-Leu-Asn-Leu-Val-Thr-Arg-Gln-Arg-Tyr-Lys(MPA)-
CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Lys(Aloc)-OH, Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Trt)-OH, Fmoc-Arg(Pb)-OH, Fmoc-Leu-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Tit)-OH, Fmoc-Leu-OH, Fmoc-Glu(tBu)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. The following protected amino acids were sequentially added to resin: They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold (0-4° C.) Et₂O (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound III

(SEQ ID NO: 4)

MPA-Ile-Lys-Pro-Glu-Ala-Pro-Gly-Glu-Asp-Ala-Ser-
Pro-Glu-Glu-Leu-Asn-Arg-Tyr-Tyr-Ala-Ser-Leu-Arg-
His-Tyr-Leu-Asn-Leu-Val-Thr-Arg-Gln-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pb)-OH, Fmoc-Gln(Tit)-OH, Fmoc-Arg(Pbf)-OH, Fmoc-Thr(tBu)-OH, Fmoc-Val-OH, Fmoc-Leu-OH, Fmoc-Asn(Tit)-OH, Fmoc-Leu-OH, Fmoc-Tyr(tBu)-OH, Fmoc-His(Trt)-OH, Fmoc-Arg(Pb)-OH, Fmoc-Leu-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Tit)-OH, Fmoc-Leu-OH, Fmoc-Glu(tBu)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Fmoc-Ile-OH MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The peptide was cleaved from the resin using 85% TFA/5% TIS/5% thioanisole and 5% phenol, followed by precipitation by dry-ice cold (0-4° C.) Et₂O (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound IV

(SEQ ID NO: 6)

Ile-Lys-Pro-Glu-Ala-Pro-Gly-Glu-Asp-Ala-Ser-Pro-
Glu-Glu-Leu-Asn-Arg-Tyr-Tyr-Ala-Ser-Lys(MPA)-Arg-
His-Tyr-Leu-Asn-Leu-Val-Thr-Arg-Gln-Arg-Tyr-CONH₂

Step 1: Solid phase peptide synthesis of the DAC derivative on a 100 mmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Tit)-OH, Fmoc-Arg(Pbf)-OH, Fmoc-Thr(tBu)-OH, Fmoc-Val-OH, Fmoc-Leu-OH, Fmoc-Asn(Tit)-OH, Fmoc-Leu-OH, Fmoc-Tyr(tBu)-OH, Fmoc-His(Trt)-OH, Fmoc-Arg(Pbf)-OH, Fmoc-Lys(Aloc)-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Trt)-OH, Fmoc-Leu-OH, Fmoc-Glu(tBu)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound V

(SEQ ID NO: 7)

Ile-Lys-Pro-Glu-Ala-Pro-Gly-Glu-Asp-Ala-Ser-Pro-
Glu-Glu-Leu-Asn-Arg-Lys(MPA)-Tyr-Ala-Ser-Leu-Arg-
His-Tyr-Leu-Asn-Leu-Val-Thr-Arg-Gln-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Trt)-OH, Fmoc-Arg(Pb)-OH, Fmoc-Thr(tBu)-OH, Fmoc-Val-OH, Fmoc-Leu-OH, Fmoc-Asn(Tit)-OH, Fmoc-Leu-OH, Fmoc-Tyr(tBu)-OH, Fmoc-His(Tit)-OH, Fmoc-Arg(Pbf)-OH, Fmoc-Leu-OH, Fmoc-Ser(tBu)-OH, Fmoc-Ala-OH, Fmoc-Tyr(tBu)-OH, Fmoc-Lys(Aloc)-OH, Fmoc-Arg(Pbf) OH, Fmoc-Asn(Trt)-OH, Fmoc-Leu-OH, Fmoc-Glu(tBu)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound VI

(SEQ ID NO: 8)

Ile-Lys-Pro-Glu-Ala-Pro-Gly-Glu-Asp-Ala-Ser-Pro-
Glu-Glu-Lys(MPA)-Asn-Arg-Tyr-Tyr-Ala-Ser-Leu-Arg-
His-Tyr-Leu-Asn-Leu-Val-Thr-Arg-Gln-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Trt)-OH, Fmoc-Arg(Pbf)-OH, Fmoc-Leu-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Trt)-OH, Fmoc-Lys(Aloc)-OH, Fmoc-Glu(tBu)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound VII

(SEQ ID NO: 9)

Ile-Lys-Pro-Glu-Ala-Pro-Gly-Glu-Asp-Ala-Ser-Pro-
Glu-Lys(MPA)-Leu-Asn-Arg-Tyr-Tyr-Ala-Ser-Leu-Arg-
His-Tyr-Leu-Asn-Leu-Val-Thr-Arg-Gln-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Tit)-OH, Fmoc-Arg(Pbf)-OH, Fmoc-Leu-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Trt)-OH, Fmoc-Leu-OH, Fmoc-Lys(Aloc)-OH, Fmoc-Glu(tBu)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound VIII

(SEQ ID NO: 10)

Ile-Lys-Pro-Glu-Ala-Pro-Gly-Glu-Asp-Ala-Ser-Pro-
Lys(MPA)-Glu-Leu-Asn-Arg-Tyr-Tyr-Ala-Ser-Leu-Arg-
His-Tyr-Leu-Asn-Leu-Val-Thr-Arg-Gln-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc-Asn(Tit)-OH, Fmoc-Leu-OH, Fmoc-Tyr(tBu)-OH, Fmoc-His(Trt)-OH, Fmoc-Arg(Pbf)-OH, Fmoc-Leu-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Trt)-OH, Fmoc-Leu-OH, Fmoc-Glu(tBu)-OH, Fmoc-Lys(Aloc)-OH, Fmoc-Pro-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound IX

(SEQ ID NO: 11)

Ile-Lys-Pro-Glu-Ala-Pro-Gly-Glu-Asp-Ala-Ser-Lys
(MPA)-Glu-Glu-Leu-Asn-Arg-Tyr-Tyr-Ala-Ser-Leu-Arg-
His-Tyr-Leu-Asn-Leu-Val-Thr-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Trt)-OH, Fmoc-Arg(Pb)-OH, Fmoc-Leu-OH, Fmoc-Ser(tBu)-OH, Fmoc-Ala-OH, Fmoc-Tyr(tBu)-OH, Fmoc-Tyr(tBu)-OH, Fmoc-Arg(Pbf)-OH, Fmoc-Asn(Trt)-OH, Fmoc-Leu-OH, Fmoc-Glu(tBu)-OH, Fmoc-Glu(tBu)-OH, Fmoc-Lys(Aloc)-OH, Fmoc-Ser(tBu)-OH, Fmoc-Ala-OH, Fmoc-Asp(tBu)-OH, Fmoc-Glu(tBu)-OH, Fmoc-Gly-OH, Fmoc-Pro-OH, Fmoc-Ala-OH, Fmoc-Glu(tBu)-OH, Fmoc-Pro-OH, Fmoc-Lys(Boc)-OH, Boc-Ile-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloc) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound X

(SEQ ID NO: 12)

Ac-Ala-Ser-Leu-Arg-His-Tyr-Leu-Asn-Leu-Val-Thr-
Arg-Gln-Arg-Tyr-CONH

Step 1: Solid phase peptide synthesis of the DAC derivative on a 100 mmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Tit)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Trt)-OH, Fmoc-Arg(Pbf)-OH, Fmoc-Leu-OH, Fmoc-Ser(tBu)-OH, Fmoc-Ala-OH, Acetic Acid. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound XI

(SEQ ID NO: 12)

MPA-Ala-Ser-Leu-Arg-His-Tyr-Leu-Asn-Leu-Val-Thr-
Arg-Gln-Arg-Tyr-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc-Asn(Trt)-OH, Fmoc-Leu-OH, Fmoc-Tyr(tBu)-OH, Fmoc-His(Trt)-OH, Fmoc-Arg(Pb)-OH, Fmoc-Leu-OH, Fmoc-Ser(tBu)-OH, Fmoc-Ala-OH, MPA-OH. They were dissolved in N,N-dimethylformamide (DMF) and, according to the sequence, activated using O-benzotriazol-1-yl-N,N,N′,N′-tetramethyl-uronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The peptide was cleaved from the resin using 85% TFA/5% TIS/5% thioanisole and 5% phenol, followed by precipitation by dry-ice cold Et₂O (0-4° C.) (Step 4). The crude peptide was collected on a polypropylene sintered funnel, dried, redissolved in a 40% mixture of acetonitrile in water (0.1% TFA) and lyophilized to generate the corresponding crude material used in the purification process.

Compound XII

(SEQ ID NO: 13)

Ac-Ala-Ser-Leu-Arg-His-Tyr-Leu-Asn-Leu-Val-Thr-
Arg-Gln-Arg-Tyr-Lys(MPA)-CONH₂

Step 1: Solid phase peptide synthesis of the DAC derivative on a 100 μmole scale was performed using manual solid-phase synthesis, a Symphony Peptide Synthesizer and Fmoc protected Ramage resin. The following protected amino acids were sequentially added to resin: Fmoc-Lys(Aloc)-OH, Fmoc-Tyr(tBu)-OH, Fmoc-Arg(Pbf)-OH, Fmoc-Gln(Trt)-OH, Fmoc-Arg(Pbf)-OH, Fmoc-Thr(tBu)-OH, Fmoc-Val-OH, Fmoc-Leu-OH, Fmoc Asn(Trt)-OH, Fmoc-Leu-OH, Fmoc-Tyr(tBu)-OH, Fmoc-His(Tit)-OH, Fmoc-Arg(Pbf)-OH, Fmoc-Leu-OH, Fmoc-Ser(tBu)-OH, Fmoc-Ala-OH, Acetic Acid. They were dissolved in N,N-dimethylformamide (DMF) and, according to the sequence, activated using O-benzotriazol-1-yl-N,N,N′,N′-tetramethyluronium hexafluorophosphate (HBTU) and Diisopropylethylamine (DIEA). Removal of the Fmoc protecting group was achieved using a solution of 20% (V/V) piperidine in N,N-dimethylformamide (DMF) for 20 minutes (step 1).

Step 2: The selective deprotection of the Lys (Aloe) group was performed manually and accomplished by treating the resin with a solution of 3 eq of Pd(PPh₃)₄ dissolved in 5 mL of C₆H₆:CHCl₃ (1:1): 2.5% NMM (v:v): 5% AcOH (v:v) for 2 h (Step 2). The resin is then washed with CHCl₃ (6×5 mL), 20% AcOH in DCM (6×5 mL), DCM (6×5 mL), and DMF (6×5 mL).

Step 3: The synthesis was then re-automated for the addition of the 3-maleimidopropionic acid (Step 3). Between every coupling, the resin was washed 3 times with N,N-dimethylformamide (DMF) and 3 times with isopropanol.

Step 4: The peptide was cleaved from the resin using 85% TFA/5% TIS/5% thioanisole and 5% phenol, followed by precipitation by dry-ice cold Et₂O (0-4° C.)

### 2. Purification Procedure

Each product was purified by preparative reversed phase HPLC, using a Varian (Dynamax) preparative binary HPLC system.

Purification of all the above compounds were performed using a Phenomenex Luna 10μ phenyl-hexyl, 50 mm×250 mm column (particles 10μ) equilibrated with a water/TFA mixture (0.1% TFA in H₂O; Solvent A) and acetonitrile/TFA (0.1% TFA in CH₃CN; Solvent B). Elution was achieved at 50 mL/min by running various gradients of % B gradient over 180 min. Fractions containing peptide were detected by UV absorbance (Varian Dynamax UVD II) at 214 and 254 nm.

Fractions were collected in 25 mL aliquots. Fractions containing the desired product were identified by mass detection after direct injection onto LC/MS. The selected fractions were subsequently analyzed by analytical HPLC (20-60% B over 20 min; Phenomenex Luna 5μ phenylhexyl, 10 mm×250 mm column, 0.5 mL/min) to identify fractions with ≧90% purity for pooling. The pool was freeze-dried using liquid nitrogen and subsequently lyophilized for at least 2 days to yield a white powder.

Other suitable peptides are represented in the following sequences:

(SEQ ID NO: 14)

Tyr-Pro-Ala-Lys-Pro-Glu-Ala-Pro-Gly-Glu-Asp-Ala-
Ser-Pro-Glu-Glu-Leu-Ser-Arg-Tyr-Tyr-Ala-Ser-Leu-
Arg-His-Tyr-Leu-Asn-Leu-Val-Thr-Arg-Gln-Arg-Tyr;
and

(SEQ ID NO: 15)

Tyr-Pro-Ile-Lys-Pro-Glu-Ala-Pro-Gly-Glu-Asp-Ala-
Ser-Pro-Glu-Glu-Leu-Asn-Arg-Tyr-Tyr-Ala-Ser-Leu-
Arg-His-Tyr-Leu-Asn-Leu-Leu-Thr-Arg-Pro-Arg-Tyr.

### 3. Table of Products

TABLE 1
List of the various peptides prepared
together with their molecular weight

| Compound no: | Theoretical M.W. | Actual M.W. |
|---|---|---|
| I | 4049.5 | 4049.5 |
| II | 4328.8 | 4328.6 |
| III | 4200.6 | 4200.0 |
| IV | 4255.5 | 4257.1 |
| V | 4212.2 | 4214.1 |
| VI | 4197.2 | 4199.0 |
| VII | 4229.1 | 4231.5 |
| VIII | 4239.2 | 4241.6 |
| IX | 4255.2 | 4257.5 |
| X | 1931.2 | 1930.7 |
| XI | 2185.5 | 2185.0 |
| XII | 2210.5 | 2210.1 |

### 4. Flow Diagram for Each Compound

A) Identical synthetic schemes, as exemplified in the flow diagram below, were employed for all stabilized DAC™. Of course, for the natives the Aloc removal step along with the addition step of AEEA and\or MPA were omitted.

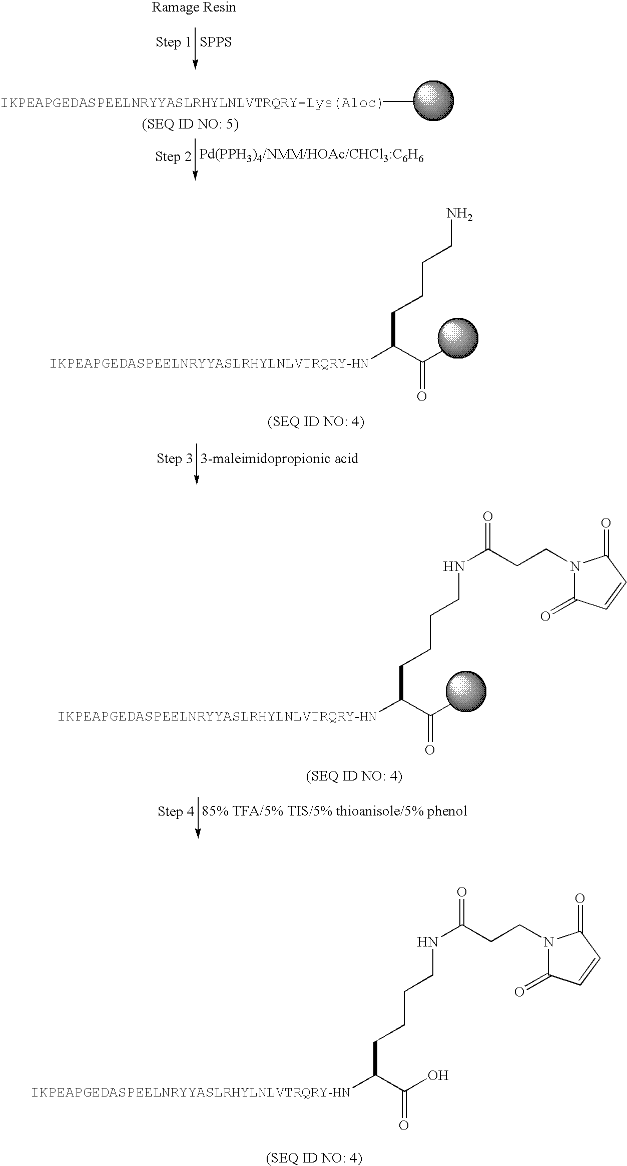

### Direct Synthesis

Alternative Synthesis of Compound III and isolation of Compound XIII

PYY₃₋₃₆ (human) is a 34 amino acids peptide. From the sequence, the N-terminal and lysine residue (in position 2) can be modified by direct attachment of the DAC group. Since the peptide is not very soluble in DMF, it has to be treated with TFA to be dissolved and then neutralized by NMM. Thus, the reaction has to be in the TFA/NMM buffer system. However, both amino groups in N-terminal and lysine show the same reactivity towards MPA-OSu under the buffer system. With 1 equivalent of MPA-OSu in the TFA/NMM system, the reaction produced four different products. The differences between these products are the position of the MPA on the sequence and the number of MPA attached to the sequence. Two positional isomers of having a single MPA group (MPA-PYY) have been obtained as major products and two positional isomers having two MPA groups ((MPA)₂ PYY and cyclization)) have been minor products. These four products were separated by HPLC. The positional isomers bearing a single MPA have been isolated to give MPA-PYY positional isomer-1 (Compound XIII) and positional isomer-2 (Compound II) in 27.8 and 15.2% yield respectively (see the following scheme). The starting material PYY was also recovered (37.6% recovery).

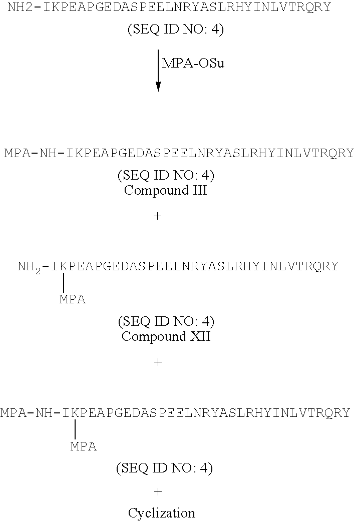

### Compounds XIV and XV

In the same way, PYY can react with excess MPA-OA-OpNP for overnight to give two positional isomers having a single MPA group MPA-OA-PYY isomer-1 (Compound XIV, 19% yield) and MPA-OA-PYY isomer-2 (Compound XV, 17.2% yield). In this cases MPA-OA-OpNP ester is less reactive and thus a large excess reagent is required for the reaction to occur. The minor products are still cyclization and (MPA-OA)₂PYY.

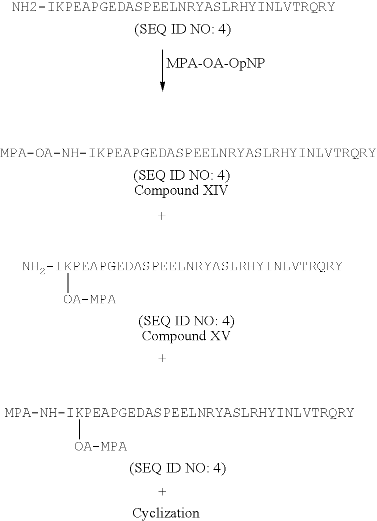

PYY (100 mg) was dissolved in DMF (5 mL) in the presence of TFA (25 μL) with the help of sonication. Then NMM (100 μL) was added followed by addition of MPA-OSu (5.6 mg). The reaction was stirred at room temperature for 2.5 h. The reaction was quenched by addition of AcOH (1 mL). The DMF solution was diluted with water to 20 mL. The products were separated by semi-preparative HPLC column (3 injections) to give PYY (37.6 mg), MPA-PYY isomer-1 (Compound XII, 27.8 mg) and PMA-PYY isomer-2 (Compound XIII, 15.2 mg).

PYY (50 mg) was dissolved in DMF (5 mL) in the presence of TFA (25 μL). NMM (100 μL) was then added followed by MPA-OA-OpNP (50 mg). The reaction was stirred for 16 h at room temperature. The linker was removed by addition of ether and the solution removed after centrifugation. The precipitate was dissolved in water and injected to semi-preparative HPLC to give MPA-OA-PYY isomer-1 (Compound XIV, 9.5 mg) and MPA-OA-PYY isomer-2 (Compound XV, 8.6 mg).

Compound XVI

Compound III was solubilized in nanopure water at a concentration of 10 mM then diluted to 1 mM into a solution of HSA (25%, Cortex-Biochem, San Leandro, Calif.). The samples were then incubated at 37° C. for 30 min. Prior to purification, the conjugate solution was diluted to 5% HSA in 20 mM sodium phosphate buffer (pH 7) composed of 5 mM sodium octanoate and 750 mM (NH₄)₂SO₄.

Using an ÄKTA purifier (Amersham Biosciences, Uppsala, Sweden), the conjugate was loaded at a flow rate of 2.5 ml/min onto a 50 ml column of butyl sepharose 4 fast flow resin (Amersham Biosciences, Uppsala, Sweden) equilibrated in 20 mM sodium phosphate buffer (pH 7) composed of 5 mM sodium octanoate and 750 mM (NH₄)₂SO₄. Under these conditions, Compound XVI adsorbed onto the hydrophobic resin whereas essentially all non-conjugated (unreacted) HSA eluted within the void volume of the column. The conjugate was further purified from any free (unreacted) maleimido PYY₃₋₃₆ derivative by applying a linear gradient of decreasing (NH₄)₂SO₄ concentration (750 to 0 mM) over 4 column volumes. The purified conjugate was then desalted and concentrated using Amicon® ultra centrifugal (30 kDa) filter devices (Millipore Corporation, Bedford, Mass.). Finally, the conjugate solution was immersed into liquid nitrogen, lyophilized and stored at −80° C.

Compounds XVII to XXII

Compounds XVII to XXII are all conjugates having in form of a white solid and they have been prepared according to the same manner than Compound XVI. The table below indicates from which peptides these conjugates have been prepared. Moreover, the molecular weight of each conjugate is given.

TABLE 2
Conjugates obtained from various peptides.

Mᵣ (conjugates)

| Peptides | Conjugate | Predicted | Measured |
|---|---|---|---|
| Compound III | Compound XVI | 70643 | 70639 |
| Compound XIII | Compound XXI | 70645 | 70640 |
| Compound XI | Compound XXII | 68629 | 68626 |
| Compound II | Compound XIX | 70771 | 70668 |
| Compound XII | Compound XVIII | 68654 | 68651 |
| Compound XV | Compound XVII | 70787 | 70785 |
| Compound XIV | Compound XX | 70787 | 70785 |

### Example I

### In Vitro Binding Assay: Selectivity Toward the NPY Y2 Receptor

Serially diluted test compounds (10⁻¹³M to 10⁻⁵M) were incubated for 60 minutes at 37° C. in the presence of 4.09 μg of human neuropeptide Y2 receptor expressing human KAN-TS cells and 50000 CPM of ¹²⁵I-PYY₃₋₃₆. The individual solutions were filtered (Whatman 934 A/H filters) and washed with ice-cold buffer. The filters were then placed in a gamma counter and the values reported as the percent relative to the maximum gamma emission at the zero concentration as a function of test compound concentration as shown on Table 3.

TABLE 3
Comparison of the NPY Y2 Receptor
binding of the PYY derivatives

| Peptide | Sequence | IC₅₀ (nM) |
|---|---|---|
| Compound I | PYY₃₋₃₆ | 1.17 |
| NPY(₁₃₋₃₆) control | — | 2.48 |
| Compound XVI | N-term SL PYY₃₋₃₆-HSA | 35.2 |
| | conjugate | |
| Compound XVII | N-term LL PYY₃₋₃₆-HSA | 52.4 |
| | conjugate | |
| Compound XX | N-K5 LL PYY₃₋₃₆-HSA | 40.2 |
| | conjugate | |
| Compound XIX | C-term SL PYY₃₋₃₆-HSA | >10³ |
| | conjugate | |
| Compound XVIII | C-term SL PYY₂₂₋₃₆-HSA | >10³ |
| | conjugate | |

### Example II

### In Vitro Binding Assay: Loss of Selectivity Toward the NPY Y1 Receptor

Compound I (PYY₃₋₃₆) and Compound XVI were tested so as to evaluate the preferential binding of the Y2 receptor relative to the Y1 receptor. A selective binding to the Y2 receptor ensures reduced (unwanted) side effects such as for example hypertension

TABLE 4
Comparison of the NPY Y1 Receptor
binding of the PYY derivatives

| Peptide | Sequence | IC₅₀ (nM) |
|---|---|---|
| Compound I | PYY₃₋₃₆ | 83.2 |
| NPY(human, rat) control | — | 1.09 |
| Compound XVI | N-term SL PYY₃₋₃₆-HSA | 875.9 |
| | conjugate | |

### Example III

### Food Intake in Rats Following IV Administration of DAC

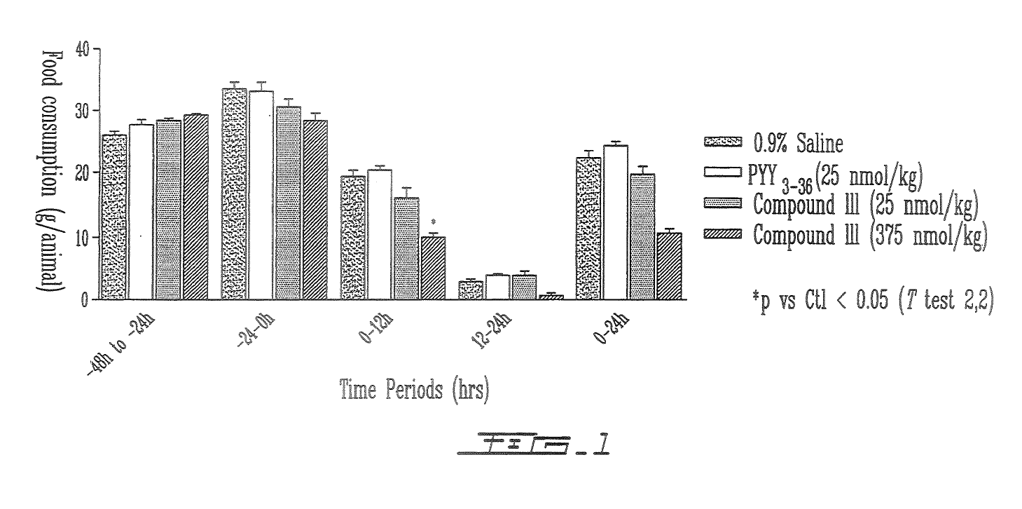

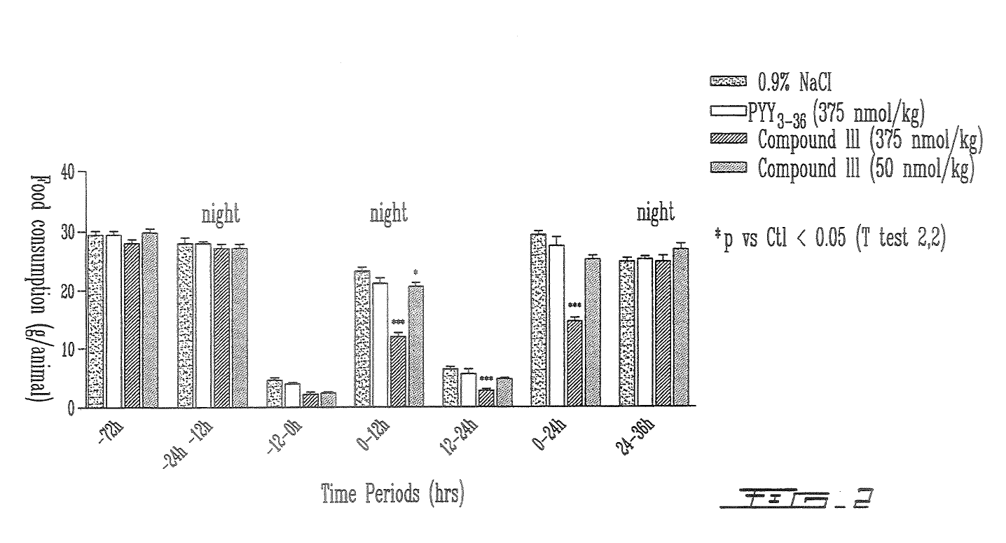

Compound I (PYY₃₋₃₆) and Compound III were injected into the tail vein of fully grown Sprague-Dawley rats. Two experiments were carried out so as to verify the influence of the concentration on Compound III of the food consumption of the animal. The food intake was measured pre and post administration (see FIGS. 1 and 2). In experiment 1 on FIG. 1, 4-500 g rats were used and in experiment 2 on FIG. 2, 2-300 g rats were used.

As it can be seen from FIGS. 1 and 2, the results showed a significant reduction in food intake over the 0-12 hour and 12-24 hour periods. The overall effect is very significant over the 0-24 hour period at the highest dose tested (375 nM/kg).

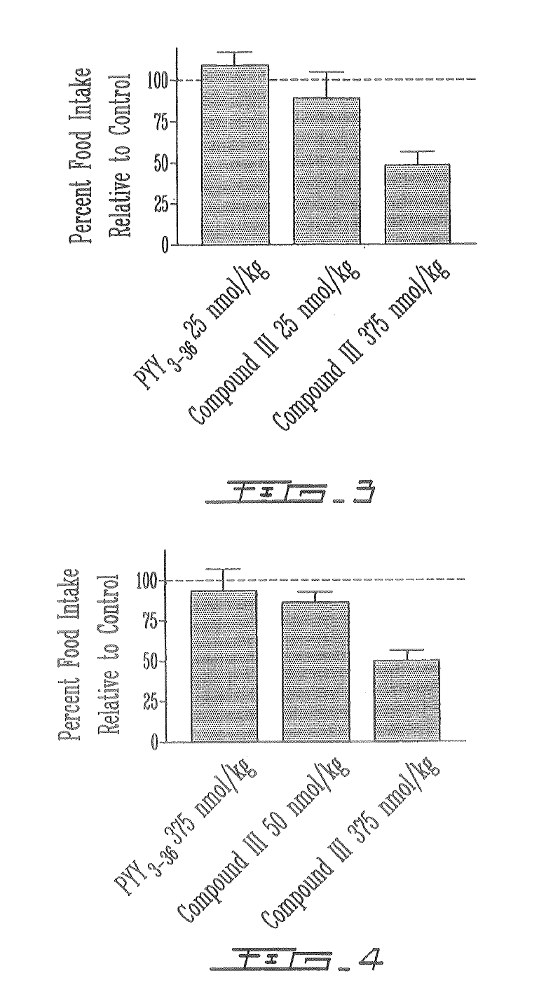

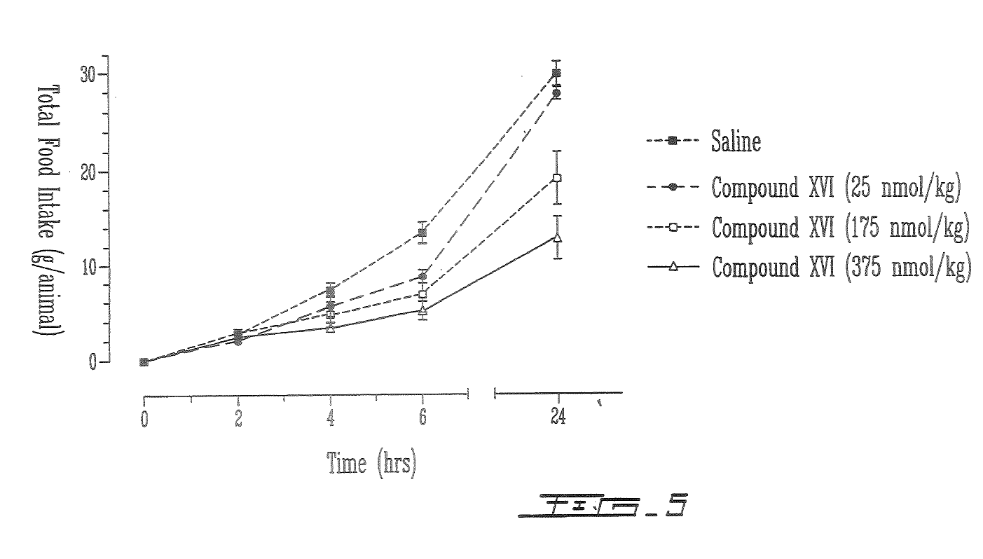

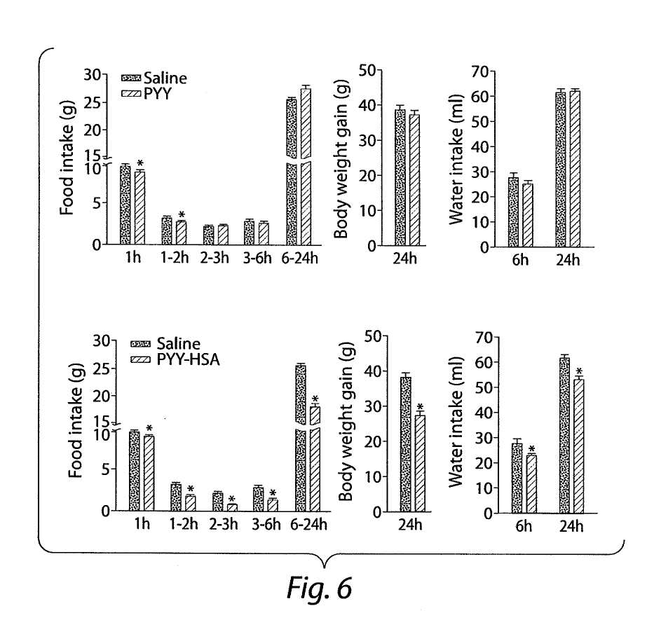

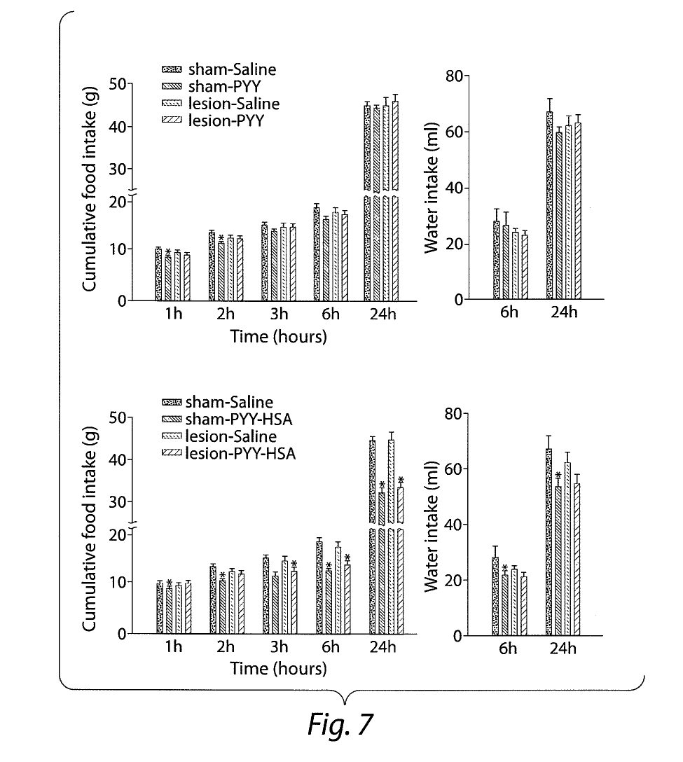

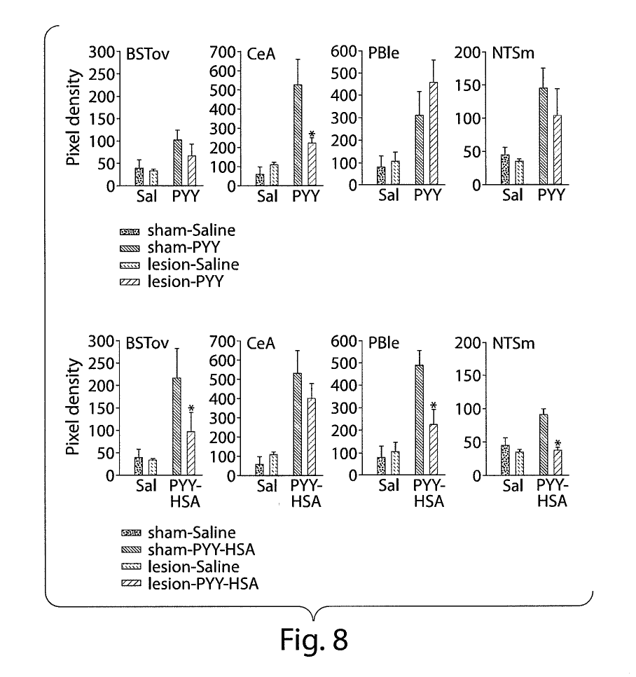

A comparison of reduction in food intake in the two experiments can easily be made by using FIGS. 3 and 4. It can be seen from FIGS. 3 and 4 that PYY₃₋₃₆ does not show reduction in food after 24 hrs at a dose of 25 nmol/kg or at a dose of 375 nmol/kg, while Compound III, at a dose of 375 nmol/kg, shows a strong effect by reducing food intake by 50% after 24 hrs. This comparison demonstrates the long lasting effect of Compound III as compared to the free peptide PYY₃₋₃₆ in vivo.

### Example IV

### Peripheral vs. Central Action of Compound III

A publication by Batterham (Nature, 2002, 418, 650-654) demonstrated the strong effect of PYY₃₋₃₆ administration into the arcuate nucleus to rats on overall food intake. The arcuate nucleus does possess a blood brain barrier and therefore no evidence was ever shown in the literature that peripheral neuropeptide Y2 receptors would have and influence on food intake. Applicant has shown that Compound XVI cannot cross the blood brain barrier (molecular weight >70 000 Da). It is known that PYY₃₋₃₆ interacts with the Y2 receptor found in the arcuate nucleus of the hypothalamus. This receptor is found behind the blood brain barrier (BBB). Nonaka et al., in an article entitled “Characterization of blood-brain barrier permeability to PYY₃₋₃₆ in the mouse” and published in J. Pharmacol. Exp. Ther. 2003, 306, 948-53, have hypothesized that the PYY must “cross the BBB” in order to be responsible for the appetite regulating activity.

The injection of Compound XVI i.p. into acclimatized Sprague-Dawley rats in a repeat of the Batterham experiment showed the results shown in FIG. 5.

According to FIG. 5, the 375 nmol/kg dose showed significant reduction in food intake at the 4 hour time point in the experiment. The results are comparable to PYY3-36 25 nmol/kg. Even though there is 15 fold more Compound XVI, pharmacokinetics of absorption will play a role in this head to head comparison. Compound XVI will peak in plasma at a later time than the short peptide. This experiment was done to compare the HSA conjugate directly to the peptide.

It has thus been demonstrated from FIGS. 3 and 4 that the compounds are very effective for treating food disorders such as obesity. In fact, the peptide (Compound III) demonstrated an activity which clearly superior than the activity of PYY₃₋₃₆. It can also be inferred from the results shown in FIG. 5 that the conjugate (Compound XVI) is prevented from crossing the blood brain barrier. In fact reduction in food intake via the PYY receptors (Y1 and Y2, which are thought to have an important role in appetite reduction) is thought to be found on the arcuate nucleus. There is a blood brain barrier separating the arcuate nucleus from plasma. The importance of this experiment is that the conjugate (Compound XVI) has a molecular mass >70 kDA. Therefore, this compound does not cross the blood brain barrier. It can thus be assumed that the PYY receptors involved in the reduction of food intake are found peripherally.

As demonstrated in Kratz et al. J. Med. Chem. 2002, 45, 5523-33, when a compound containing a reactive maleimide group such as compound III is injected in a patient, this compound will be eventually covalently bonded to albumin, thereby being converted into Compound XVI. It can thus be said from FIGS. 1 to 5 that the enhanced activity of Compound III with respect to the activity of PYY₃₋₃₆ is due to the fact that Compound III is prevented from crossing the blood brain barrier when the latter is covalently bonded to HSA (converted into Compound XVI).

It has thus been surprisingly noted that by preventing a PYY peptides or derivative thereof from crossing the blood brain barrier, an enhanced anti-obesity activity of this peptide is observed as compared to the peptide alone.

### Example V

### Assessment of Brain Structures Involved with Circulating PYY Signals

Male Wistar rats were either sham operated or subjected to electrolytic lesions of the AP, SFO or both. Two weeks were allowed for recovery after surgeries while body weight, food and water intake were measured daily. Then rats, which were fasted for 24 hours, were subcutaneously injected with anorexigenic doses of PYY-HAS, PYY or saline (for controls) following which free access to food was reallowed. Food and water intake were measured for 24 hours following drug injections. Both peptides induced a reduction in food intake during refeeding. The anorexigenic effect of PYY was detected only during the first two hours of refeeding (with a maximum of 1 hour) while the effect of PYY-HSA was more persistent (up to 24 hours after the injection). Moreover, PYY-HAS induced a reduction in water intake and body weight gain after food deprivation but PYY did not. However, the anorexigenic effect of both peptides was delayed in AP-SFO-lesioned rats, 1 hour in PYY treated rats and 2 hours in PYY-HAS treated rats.

The long lasting effect of the PYY conjugate versus the short term effect of PYY demonstrates its effectiveness as an anorectic drug. Although temporary, the delay induced in the double lesioned rat suggests a potential role for AP and SFO in the central action of PYY. However, other mechanisms seem to be necessary for a complete anorectic effect of PYY.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed:

1. A method of reducing water intake and body weight gain after food deprivation in a subject, comprising: administering to a subject a PYY peptide-albumin conjugate comprising albumin, a PYY peptide or derivative thereof and a maleimido-containing group attached, with or without a linking group, to an amino acid of the PYY peptide to form a modified PYY peptide, wherein the reactive group forms a covalent bond with a thiol of albumin, to thereby reduce water intake and body weight gain in the subject.

2. The method of claim 1, wherein the subject has been on a diet of reduced food intake for at least one day up to one month.

3. The method of claim 1, wherein the PYY peptide-albumin conjugate is administered before the intake of food after the subject has been on a diet of reduced food intake.

4. The method of claim 1, wherein the PYY peptide is PYY₃₋₃₆.

5. The method of claim 1, wherein the PYY peptide derivative is PYY₂₂₋₃₆.

6. The method of claim 1, wherein the modified PYY peptide or derivative thereof is selected from SEQ ID NOs: 1-15.

7. The method of claim 6, wherein the modified PYY peptide or derivative thereof comprises SEQ ID NO:4, 6, 7, 8, 9, 10, 11, 12 or 13.

8. The method of claim 1, wherein the conjugate comprises a linking group and the linking group that attaches to the reactive group and the amino acid of the PYY peptide or derivative thereof is selected from the group consisting of (2-amino)ethoxy acetic acid (AEA), ethylenediamine (EDA), amino ethoxy ethoxy succinimic acid (AEES), 2-[2-(2-amino)ethoxy)]ethoxy acetic acid (AEEA), AEEA-AEEA, —NH.sub.2-(CH₂)ₙ—COOH where n is an integer from 1 to 20; one or more alkyl chains (C₁-C₁₀) saturated or unsaturated which optionally comprises oxygen, nitrogen or sulfur atoms motifs, glycine, 3-aminopropionic acid (APA), 8-aminooctanoic acid (OA), 4-aminobenzoic acid (APhA), and combinations thereof.

9. The method of claim 1, wherein the reactive group that forms a covalent bond with the thiol of albumin is selected from gamma-maleimide-butyralamide (GMBA), beta-maleimidopropionic acid (MPA) and alpha-maleimidoacetic acid (MAA).

10. The method of claim 1, wherein the subject has a tonicity disorder or condition.

11. The method of claim 11, wherein the subject is hypotonic.

12. The method of claim 1, wherein the subject is obese.

13. A method of reducing food intake during periods in between a meal, comprising: administering to a subject a PYY peptide-albumin conjugate comprising albumin, a PYY peptide or derivative thereof and a maleimido-containing group attached, with or without a linking group, to an amino acid of the PYY peptide to form a modified PYY peptide, wherein the reactive group forms a covalent bond with a thiol of albumin, to thereby reduce food intake in between meals by the subject.

14. The method of claim 13, wherein the period in between meals during which food intake is reduced is 3 to 8 hours.

15. The method of claim 13, wherein the PYY peptide is PYY₃₋₃₆.

16. The method of claim 13, wherein the PYY peptide derivative is PYY₂₂₋₃₆.

17. The method of claim 13, wherein the modified PYY peptide or derivative thereof is selected from SEQ ID NOs: 1-15.

18. The method of claim 17, wherein the modified PYY peptide or derivative thereof comprises SEQ ID NO:4, 6, 7, 8, 9, 10, 11, 12 or 13.

19. The method of claim 13, wherein the conjugate comprises a linking group and the linking group that attaches to the reactive group and the amino acid of the PYY peptide or derivative thereof is selected from the group consisting of (2-amino)ethoxy acetic acid (AEA), ethylenediamine (EDA), amino ethoxy ethoxy succinimic acid (AEES), 2-[2-(2-amino)ethoxy)]ethoxy acetic acid (AEEA), AEEA-AEEA, —NH.sub.2-(CH₂)ₙ—COOH where n is an integer from 1 to 20; one or more alkyl chains (C₁-C₁₀) saturated or unsaturated which optionally comprises oxygen, nitrogen or sulfur atoms motifs, glycine, 3-aminopropionic acid (APA), 8-aminooctanoic acid (OA), 4-aminobenzoic acid (APhA), and combinations thereof.

20. The method of claim 13, wherein the reactive group that forms a covalent bond with the thiol of albumin is selected from gamma-maleimide butyralamide (GMBA), betamaleimidopropionic acid (MPA) and alpha-maleimidoacetic acid (MAA).


SEQUENCE LISTING (15 sequences)

SEQ ID NO: 1 - protein, 37 aa, Artificial Sequence
  Description of Artificial Sequence Synthetic peptide
XPXXPXXPGXXXXXXXXXXYXXXLXXYXNXXTXXXXX

SEQ ID NO: 2 - protein, 12 aa, Artificial Sequence
  Description of Artificial Sequence Synthetic peptide
AXXRXXXNXXXR

SEQ ID NO: 3 - protein, 34 aa, Artificial Sequence
  Description of Artificial Sequence Synthetic peptide
YPIKPEAPGEDDEELNRYYASLRHYLNLVTRQRY

SEQ ID NO: 4 - protein, 34 aa, Artificial Sequence
  Description of Artificial Sequence Synthetic peptide
IKPEAPGEDASPEELNRYYASLRHYLNLVTRQRY

SEQ ID NO: 5 - protein, 35 aa, Artificial Sequence
  Description of Artificial Sequence Synthetic peptide
IKPEAPGEDASPEELNRYYASLRHYLNLVTRQRYK

SEQ ID NO: 6 - protein, 34 aa, Artificial Sequence
  Description of Artificial Sequence Synthetic peptide
IKPEAPGEDASPEELNRYYASKRHYLNLVTRQRY

SEQ ID NO: 7 - protein, 34 aa, Artificial Sequence
  Description of Artificial Sequence Synthetic peptide
IKPEAPGEDASPEELNRKYASLRHYLNLVTRQRY

SEQ ID NO: 8 - protein, 34 aa, Artificial Sequence
  Description of Artificial Sequence Synthetic peptide
IKPEAPGEDASPEEKNRYYASLRHYLNLVTRQRY

SEQ ID NO: 9 - protein, 34 aa, Artificial Sequence
  Description of Artificial Sequence Synthetic peptide
IKPEAPGEDASPEKLNRYYASLRHYLNLVTRQRY

SEQ ID NO: 10 - protein, 34 aa, Artificial Sequence
  Description of Artificial Sequence Synthetic peptide
IKPEAPGEDASPKELNRYYASLRHYLNLVTRQRY

SEQ ID NO: 11 - protein, 34 aa, Artificial Sequence
  Description of Artificial Sequence Synthetic peptide
IKPEAPGEDASKEELNRYYASLRHYLNLVTRQRY

SEQ ID NO: 12 - protein, 15 aa, Artificial Sequence
  Description of Artificial Sequence Synthetic peptide
ASLRHYLNLVTRQRY

SEQ ID NO: 13 - protein, 16 aa, Artificial Sequence
  Description of Artificial Sequence Synthetic peptide
ASLRHYLNLVTRQRYK

SEQ ID NO: 14 - protein, 36 aa, Artificial Sequence
  Description of Artificial Sequence Synthetic peptide
YPAKPEAPGEDASPEELSRYYASLRHYLNLVTRQRY

SEQ ID NO: 15 - protein, 36 aa, Artificial Sequence
  Description of Artificial Sequence Synthetic peptide
YPIKPEAPGEDASPEELNRYYASLRHYLNLLTRPRY